SEQUENCE LISTING (4 sequences)

SEQ ID NO: 1 - DNA, 21 bp, Artificial
  siRNA, sense strand
ggaugaguccuccauguuctt

SEQ ID NO: 2 - DNA, 21 bp, Artificial
  siRNA, anti-sense strand
gaacauggaggacucaucctt

SEQ ID NO: 3 - DNA, 20 bp, Artificial
  siRNA, sense strand
agcucguucccaaagagcuu

SEQ ID NO: 4 - DNA, 21 bp, Artificial
  siRNA, anti-sense strand
uuucgagcaaggguuuucucg

(19) United States
(12) Patent Application Publication
(10) Pub. No.: US 2007/0161592 A1
(43) Pub. Date: Jul. 12, 2007

(54) Method of treating fear

(76) Inventor: Min Zhuo, Toronto (CA)

Correspondence Address: GOWLING, LAFLEUR HENDERSON LLP, ONE MAIN STREET WEST, HAMILTON, ON, L8P 4Z5, CA

(21) Appl. No.: 11/520,591

(22) Filed: Sep. 14, 2006

Related U.S. Application Data
(60) Provisional application No. 60/716,993, filed on Sep. 14, 2005.

(51) Int. Cl. A61K 48/00

(52) U.S. Cl. 514/44

(57) ABSTRACT

A method of treating conditions associated with fear memory in a mammal is provided. The method comprises inhibiting NR2B receptors in the ACC of the animal.

### FIELD OF THE INVENTION

The present invention relates to a method of treating fear in which NMDA receptors in the anterior cingulated cortex (ACC) are targeted. In particular, the present invention relates to a method of treating fear-related conditions in which certain NMDA receptors are inhibited in the ACC.

### BACKGROUND OF THE INVENTION

Glutamate NMDA receptors (NMDARs) are required for the synaptic plasticity associated with the mechanisms of learning and memory (Bliss and Collingridge, 1993; Malenka and Nicoll, 1993). Native NMDARs are composed of NR1, NR2 (A, B, C, and D) and NR3 (A and B) subunits. The formation of functional NMDARs requires a combination of NR1, an essential channel-forming subunit, and at least one NR2 subunit. It is known that the NR2A and NR2B subunits predominate in the forebrain where they determine many of the functional properties of NMDARs (Loftis and Janowsky, 2003; Monyer et al., 1994). Moreover, NMDARs of various subunit compositions may occur during early development and in different brain areas (Arrigoni and Greene, 2004; Monyer et al., 1994; Munoz et al., 1999; Ritter et al., 2002; Sheng et al., 1994).

In central synapses, NMDAR activation is required for LTP induction (Bear and Kirkwood, 1993; Bliss and Collingridge, 1993; Lisman, 2003), and both NR2A- and NR2B-dependent signaling pathways are believed to be involved in hippocampal long term synaptic plasticity in adult mice (Kiyama et al., 1998; Kohr et al., 2003; McHugh et al., 1996). For example, studies of transgenic mice overexpressing NMDAR NR2B in the adult forebrain and KIF17 transgenic mice with upregulated NR2B expression demonstrate the important contribution made by NR2B subunits to hippocampal LTP and behavioral learning (Tang et al., 1999; Wong et al., 2002). A recent study suggested that hippocampal LTP is mediated by NMDARs containing the NR2A but not NR2B subunit (Liu et al., 2004). This finding suggests that the NMDA NR2B receptors in the hippocampus may not contribute to learning-related synaptic potentiation; however, no behavioral study has demonstrated the inhibitory effect that NR2B antagonists may have on learning when injected locally into the hippocampus.

Forebrain structures, including the anterior cingulate cortex (ACC), are thought to be important for higher brain function, and neuronal activity in these areas play important roles in emotion, learning and memory (Devinsky et al., 1995; Wiltgen et al., 2004; Zhuo, 2002). Moreover, activity-dependent gene imaging and regional inactivation studies have shown that the ACC is involved in remote fear and spatial memory (Frankland et al., 2004; Maviel et al., 2004). Although CaMKII is suggested to be required for LTP and permanent memory in the ACC (Frankland et al., 2001), the synaptic mechanisms underlying LTP and memory in the prefrontal cortex have been far less investigated than in hippocampal synapses. In terms of the acquisition of fear memory, the majority of previous studies have been done in the amygdala (Rodrigues et al., 2004). Even though neurons in the prefrontal cortex have projections to the amygdala (Cassell and Wright, 1986) and the role of the prefrontal cortex in fear extinction has been reported (Milad and Quirk, 2002; Santini et al., 2004), only a few conflicting results are available on the contributions of the prefrontal and/or ACC on fear memory acquisition (Gao et al., 2004; Han et al., 2003; Johansen and Fields, 2004; Tang et al., 2005).

Given that the role of the ACC in fear memory is currently unclear, it would be desirable to study further this region of the brain in an attempt to develop methods of treating fear-related conditions.

### SUMMARY OF THE INVENTION

It has now been found that selective inhibition of NR2B receptors in the ACC impairs the formation of early contextual fear memory. Thus, inhibition of NR2B in the ACC is useful to treat fear-related health conditions due to the prevention of contextual fear memory development that occurs when NR2B receptors are inhibited in the ACC.

Thus, in one aspect of the present invention, there is provided a method of treating in a mammal a condition associated with contextual fear memory comprising the step of selectively inhibiting NR2B receptors in the anterior cingulate cortex.

In another aspect of the present invention, an article of manufacture is provided comprising a labeled container within which is a composition comprising an NR2B inhibitor suitable for the selective inhibition of NR2B in the ACC of a mammal, the label indicating that the composition is suitable to treat conditions associated with contextual fear memory.

These and other aspects of the present invention are described by reference to the following figures in which:

### BRIEF DESCRIPTION OF THE DRAWINGS

FIG. 1 illustrates results evidencing that LTP is induced by postsynaptic NMDA receptor activation in the ACC including current-clamp recordings (A) to identify pyramidal neurons (upper) and interneurons (bottom) by current injections of −100, 0, and 100 pA. A labeled pyramid-like neuron is shown on the top right. RP: resting membrane potential; (B) LTP was induced in pyramidal neurons (n=15) in adult ACC by the pairing training protocol (indicated by an arrow). The insets show averages of 6 EPSCs 5 min before and 25 min after the pairing training (arrow). The dashed line indicates the mean basal synaptic responses; (C) An example showing the long-lasting synaptic potentiation. Pairing training is indicated by an arrow; and (D) Basic synaptic transmission showing no change during recording without applying pairing training. The insets show averages of 6 EPSCs at the time points of 5 (pre) and 35 (post) min during the recording; and (E/F) show that LTP was completely blocked by applications of AP-5 (n=7) and BAPTA (n=7) in the intracellular solution. The insets show averages of 6 EPSCs 5 min before and 25 min after the pairing training (arrow). The dashed line indicates the mean basal synaptic responses;

FIG. 2 shows that paired pulse facilitation was not changed during LTP in the ACC (A). Paired pulse facilitation (PPF: the ratio of EPSC2/EPSC1) was recorded with a 50 ms interval throughout LTP recordings (n=5). LTP was induced by pairing training. The dashed line indicates the mean PPF ratio; LTP (shown as EPSC1 amplitude) was induced (B) in ACC neurons (n=5). The insets show averages of 6 EPSCs 5 min before and 25 min after the pairing procedure (arrow). The dashed line indicates the mean basal synaptic responses.

FIG. 3 shows the contributions of the NR2A and NR2B subunits to the induction of LTP in the ACC. (A) LTP induced by the pairing training was partially depressed by 0.1 μM (n=7) or 0.4 μM (n=9) NVP-AAM077; (B) LTP was partially depressed by 0.3 μM (n=5) or 3 μM (n=9) Ro25-6981; (C) LT? was partially depressed by 3 μM ifenprodil (n=7); (D) The co-application of 0.4 μM NVP-AAM077 and 0.3 μM Ro25-6981 completely blocked LTP (n=6); and (E) provides a summary of the effects of NMDAR subunit antagonists or postsynaptic injection of BAPTA on LTP. *p<0.05 compared to baseline;

FIG. 4 shows the effects of NR2A and NR2B subunit antagonists on LTP induced by two other protocols (A-C) LTP was induced by a coincidence of postsynaptic action potentials and unitary EPSPs (10 ms ahead). (A) This protocol produced significant LTP in adult ACC neurons (n=5). (B) LTP was blocked by 0.3 μM Ro25-6981 (n=5). (C) LTP was blocked by 0.4 μM NVP-AAM077 (n=5). (D-F) LTP was induced by the TBS protocol. (D) TBS induced significant LTP in the ACC of adult mice (n=5). (E) LTP was blocked by 0.3 μM Ro25-6981 (n=5). (F) LTP was blocked by 0.4 μM NVP-AAM077 (n=7);

FIG. 5 shows the effects of NR2A and NR2B subunit antagonists on NMDAR-mediated EPSCs in the ACC and hippocampus. (A) Representative traces show bath application of different doses of NVP-AAM077 or Ro25-6981 depressed total NMDAR-mediated currents in the ACC. (B) Representative traces show bath application of different doses of NVP-AAM077 depressed total NMDAR-mediated currents in the hippocampus (Left). In contrast, Ro25-6981 enhanced total NMDAR-mediated currents (right). (C) Plot of peak EPSC amplitude versus time, showing that Ro25-6981 depressed total NMDAR-mediated current in the ACC. (D) Plot of peak EPSC amplitude versus time, showing that Ro25-6981 enhanced total NMDAR-mediated current in the hippocampus. (E) Summary of the effects of NVP-AAM077 on NMDAR-mediated currents in the ACC and hippocampus. (F) Summary of the effects of Ro25-6981 on NMDAR-mediated currents in the ACC and hippocampus;

FIG. 6 provides a comparison of NR2B and NR2A-mediated EPSCs and their expression ratios in the ACC and hippocampus. (A) Representative traces in control solution, in NVP-AAM077 and a combination of NVP-AAM077 and Ro25-6981 in ACC and hippocampal neurons. (B) Percentage contribution by NVP-AAM077 (solid bars, n=6) and Ro25-6981 (open bars, n=6) sensitive EPSCs in the ACC and hippocampus. (C) Ro25-6981 sensitive EPSC scaled to the peak of NVP-AAM077 sensitive EPSC. (D) Time constant of EPSC decay (τ) versus the rising time (10-90%) for EPSCs mediated by NR2A (circle) and NR2B (triangle) in the ACC (open) and hippocampus (solid). (E) Representative Western blots of NMDAR subunit expression in total homogenates and (F) Summary of the relative expression ratios of NR2B to NR2A in total homogenates and synaptosomal membrane fractions. (G) synaptosomal membrane fractions, and of tyrosine phosphorylated subunits of the ACC and hippocampus. (H) Percentage of tyrosine phosphorylated NR2A and NR2B subunits in the ACC and hippocampus (n=7). *p<0.05, **p<0.01 versus the hippocampus;

FIG. 7 illustrates the effect of electroporation of siRNA on NR2B expression and LTP. (A) The effectiveness and specificity of NR2B siRNA electroporation. Western blots (left) were performed in tissue from siRNA-electroporated mice. Cortical tissue between the positive electroporation electrode and the injection site were dissected. Si (−): control siRNA; Si 2B: NR2B siRNA. Right column shows a summary of the western blot analysis. Data are presented as a percentage of control siRNA treated tissues. n=5, **p<0.01. (B) Traces showing sample NR2A and NR2B-mediated NMDAR currents from control siRNA (left upper) and NR2B siRNA (left down) electroporated ACC neurons. To calculate NR2B-mediated EPSCs, ACC neurons were sequentially treated with NVP-AAM077 and a combination of NVP-AAM077 and Ro25-6981. Statistical results (right) showing Ro25-6981-sensitive component in adult ACC neurons from NR2B siRNA (n=11) and control siRNA (n=5) electroporated mice. (C) LTP was induced by three different induction protocols in control siRNA electroporated neurons. (D) Smaller LTP induced in NR2B siRNA electroporated neurons when compared with control siRNA. (C and D) The insets show averages of 6 EPSCs 5 min before and 25 min after the pairing training (arrow). The dashed line indicates the basal synaptic responses;

FIG. 8 shows the effect of NR2B blockade on contextual fear memory. (A) ACC section (left upper) showing bilateral GFP-expressing neurons four days after electroporation. Cg1, anterior cingulate cortex area 1; Cg2, anterior cingulate cortex area 2. Representative coronal section (left down) showing ACC injection sites. Right column showing cannula tip placements in mice injected with Ro25-6981 (circles) or vehicle (asterisks) in the ACC. Scale bar: 300 μm. (B) Representative coronal section (left) showing hippocampal injection sites. Only one of the bilaterally injected sides is shown. Right column showing cannula tip placements in mice injected with Ro25-6981 (circles) or vehicle (asterisks) in the hippocampus. Scale bar: 300 μm. (C) The introduction of siRNA against NR2B into the adult cortex impaired contextual fear memory (open bars: EGFP only, n=8; hatched bar: EGFP+control siRNA, n=10; crossed bar: electric shock only: n=8; filled bar, NR2B siRNA+EGFP, n=11; t-test, **p<0.01). (D) Pharmacological blockade of NR2B in the cortex decreased contextual fear memory. Open bar: vehicle treated, n=10; solid bar: Ro25-6981 injected, n=11; *p<0.05. (E) Inhibition of NR2B in the hippocampus did not impair contextual fear memory. Open bar: vehicle treated, n=9; solid bar: Ro25-6981 treated, n=12. (F) Representative coronal section showing ACC injection sites. Scale bar: 600 μm. (G) Placement of cannulas in the rat hippocampus solid squares: vehicle; solid circles: Ro25-6981. Scale bar: 600 μm. (H) Inhibition of NR2B in the ACC impaired contextual fear memory. Open bar: vehicle treated, n=6; solid bar: Ro25-6981 treated, n=6. (I) Inhibition of NR2B in the hippocampus did not impair contextual fear memory;

FIG. 9 shows that NMDAR-mediated currents were decreased after LTP induction following pairing-training (A) & (B); and

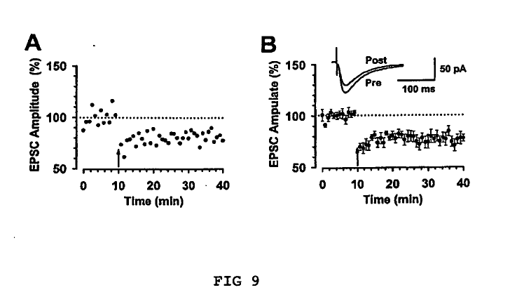

FIG. 10 illustrates the effect of high dose and two site injection of NR2B antagonist on contextual and auditory fear memory.

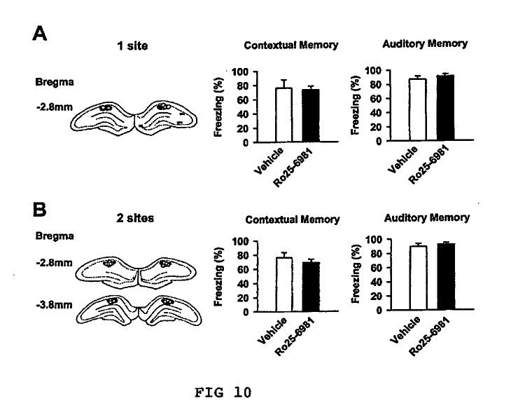

### DETAILED DESCRIPTION OF THE INVENTION

A method of treating conditions associated with contextual fear memory in a mammal is provided. The method comprises the selective inhibition of NR2B in the anterior cingulate cortex.

The terminology “condition associated with contextual fear memory” refers to a disease or abnormal state in which the existence of contextual fear memory disrupts normal day-to-day activities. Contextual fear occurs when an association between a distinctive context and an aversive event takes place in that context. A mammal that has developed contextual fear, when placed back into the context, exhibits a fear response, including for example, anxiety, depression or freezing. Thus, conditions associated with contextual fear include, but are not limited to, anxiety and depression including anxiety and fear related to chronic pain, or medical treatments including dental procedures as well as procedures necessary for diagnosis of a health condition or disease such as internal scopes and cancer treatments.

The term “mammal” refers to both human and non-human mammals.

The term “treatment” or “treating” refers to the prophylaxis, amelioration or elimination of at least one condition associated with fear memory as defined above.

In accordance with the treatment method of the present invention, selective inhibition of the NR2B subunit of NMDA receptors in the ACC results in a reduced contextual fear response as exemplified in the specific examples herein. Moreover, provided that the NR2B inhibition is selectively localized to the ACC, side effects associated with the treatment are minimal, if present at all. The term “selectively” refers to inhibition of NR2B specifically in the ACC and does not include inhibition of NR2B in other regions of the brain such as the hippocampus or the amygdala Inhibition of NR2B in the ACC need not necessarily be total inhibition in order to yield desirable effects in accordance with the invention.

In one embodiment of the present invention, NR2B in the ACC may be inhibited by administration of an NR2B antagonist or inhibitor to the ACC, provided that the NR2B is administered such that it selectively targets NR2B in the ACC. Such selectivity is obtainable by administration of the antagonist directly into the ACC. It may also be possible to administer the NR2B antagonist by modes other than direct injection into the ACC, for example orally, when administered in conjunction with a carrier compound that selectively targets the ACC.

Examples of NR2B antagonist compounds suitable for use in the present treatment method include, but are not limited to, Ro-25-6981, ifenprodil and derivatives thereof that retain NR2B inhibitory activity. Additional NR2B anatagonists may readily be identified using established protocols, for example cell line screening methods in which cells are modified by transfection to express functional mammalian NR2B in a detectable manner. The modified cell line is then grown in appropriate media in the presence of a candidate antagonist. Inhibition of NR2B activity can be monitored electrophysiologically wherein a decrease in electrophysiological activity is indicative of NR2B inhibition.

With known NR2B antagonists,such as Ro-25-6981 and ifenprodil, appropriate dosages are known and dosage forms for administration are available. For Ro-25-6981 and ifenprodil, for example, the dosage required to inhibit NR2B by direct injection into the ACC is about 1 to 10 ug, for example about 2 ug, the dosage being dependant on a number of factors such as the patient and nature of the condition being treated Suitable dosages for other NR2B antagonists can readily be determined using established in vivo protocols as well as by implementing appropriately controlled trials as one of skill in the art will appreciate.

In another embodiment, a genetic approach for inhibiting NR2B activity in the ACC may be applied, for example, siRNA technology. Application to the ACC of nucleic acid fragments such as siRNA fragments that correspond with regions in NR2B and which selectively target the NR2B gene may be used to block NR2B expression resulting in reduced contextual fear memory. Such blocking occurs when the siRNA fragments bind to the NR2B gene thereby preventing translation of the gene to yield functional receptor.

SiRNA, small interfering RNA molecules, corresponding to NR2B are made using well-established methods of nucleic acid syntheses including automated systems. Since the structure of the NR2B gene is known, fragments of RNA that correspond therewith can readily be made. Reference may be made, for example, to Sasner et al. (J. Biol. Chem. 271 (35), 21316-21322 (1996)), Kutsuwada et al. (Nature 358 (6381), 36-41 (1992)) and Sessoms-Sikes et al. (Mol. Cell. Neurosci. 29 (1), 139-147 (2005)) for NR2B nucleic acid sequence data The effectiveness of selected siRNA to block NR2B activity can be confirmed using NR2B-expressing cell lines as described above. Briefly, selected siRNA is incubated with NR2B-expressing cell line under appropriate growth conditions. Following a sufficient reaction time, i.e. for the siRNA to bind with NR2B DNA to result in decreased expression of the NR2B DNA, the reaction mixture is tested to determine if such decreased expression has occurred. Suitable siRNA will prevent processing of the NR2B gene to yield NR2B receptor. This can be detected by assaying for NR2B function in the reaction mixture, for example, ligand binding or electrophysiological activity Regions from the NR2B gene from which selective siRNA can be derived include, for example, the central region of the gene. SiRNA fragments may be designed according to the Tuschl design rule in which target sites begin with AA, include 3′-UU overhangs for both the sense and antisense SiRNA strands, have approximately 50% G/C content and are 50-100 nucleotides downstream of the start codon. siRNA designed based on the Tuschl rule may be more efficient in inhibition of targeted mRNA expression.

Examples of suitable siRNA fragments for use in the present method include, but are not limited to, sense strand 5′-GGAUGAGUCCUCCAUGUUCtt-3′ and antisense strand 5′-GAACAUGGAGGACUCAUCCtt-3′, and functionally equivalent strands thereof; sense strand 5′-AGCUCGUUCCCAAAGAGCUU-3′ and anti-sense strand 3′-UUUCGAGCAAGGGUUUUCUCG-5′, and functionally equivalent strands thereof For the purposes of the present invention, the term “functionally equivalent” as it is used herein with respect to an siRNA fragment means an siRNA fragment in which the nucleotide sequence is modified from a native siRNA sequence, but the modified fragment retains the ability to bind to the targeted NR2B gene.

It will be appreciated by one of skill in the art that functionally equivalent siRNA fragments useful in the present method may be derived from native siRNA fragments, i.e. siRNA fragments corresponding with a region of the NR2B gene such as those identified above. Modifications that may be made to native siRNA fragments to render a functionally equivalent variant siRNA strand include, for example, addition, deletion or substitution of one or more of the nucleotide bases therein, provided that the modified siRNA retains it ability to bind to the targeted NR2B gene.

Selected siRNA fragments may additionally be modified to incorporate desired properties such as increased stability. For example, siRNA fragments may be modified to include terminal protecting groups such as poly-A tails.

Synthetic siRNA can be administered to a patient for delivery to a target site using techniques established in the art including cation lipid-mediated transfection. In this regard, suitable cationic lipids include compounds such as oligofectamine and DOTAP. SiRNA may also be administered using a polycation-based gene delivery system such as a PEI conjugate. As one of skill in the art will appreciate, protocol associated with the administration of siRNA to block NR2B expression can be determined using techniques known in the art such as those described in Tan et al. Gene Therapy (2005) 12, 59-66.

In another aspect, an article of manufacture is provided. The article includes labeling and contains a composition suitable to inhibit or block NR2B in the ACC. The labeling indicates that the composition is useful to treat conditions associated with contextual fear memory. The article of manufacture may be any container suitable to retain the composition including, for example, a carton, bottle, wrap, tube or box.

As described above the composition suitable to inhibit or block NR2B may comprise a synthetic NR2B antagonist. Alternatively, the composition may comprise siRNA directed to NR2B, and capable of inhibiting the expression thereof

Embodiments of the invention are described in the following specific examples which are not to be construed as limiting.

### EXAMPLE 1

Experimental Procedures

Animals 6-8-week-old C57BL/6 male mice and male Sprague Dawley rats were used. All animals were housed under a 12:12 light cycle with food and water provided ad libitum. The Animal Care and Use Committee of University of Toronto approved the animal protocols.

Slice Preparation

Coronal brain slices (300 μM) from 6-8-week-old C57BL/6 mice, containing ACC or hippocampus, were prepared using standard methods (Wei et al., 2001). Slices were transferred to a submerged recovery chamber containing oxygenated (95% O₂ and 5% CO₂) artificial cerebrospinal fluid (ACSF) (in mM: 124 NaCl, 4.4 KCl, 2 CaCl₂, 1 MgSO₄, 25 NaHCO₃, 1 NaH₂PO₄, 10 glucose) at room temperature for at least 1 h.

Whole-Cell Recordings

Experiments were performed in a recording chamber on the stage of an Axioskop 2FS microscope with infrared DIC optics for visualizing whole-cell patch clamp recordings. EPSCs were recorded from layer II-III neurons using an Axon 200B amplifier (Axon Instruments, CA) and stimulations were delivered using a bipolar tungsten stimulating electrode placed in layer V of the ACC. In hippocampal slices, EPSCs were evoked by stimulating the Schaffer collateral-commissural pathway. EPSCs were induced by repetitive stimulations at 0.02 Hz and neurons were voltage clamped at −70 mV. The recording pipettes (3-5 MΩ) were filled with solution containing (mM) 145 K-gluconate, 5 NaCl, 1 MgCl₂, 0.2 EGTA, 10 HEPES, 2 Mg-ATP, and 0.1 Na₃-GTP (adjusted to pH 7.2 with KOH). After obtaining stable EPSCs for 10 min, three kinds of LTP induction paradigms were used within 12 min after establishing the whole-cell configuration to prevent wash out effect on LTP induction (Tsvetkov et al., 2002). The first protocol involved paired presynaptic 80 pulses at 2 Hz with postsynaptic depolarization at +30 mV (referred to as pairing training). The second involved paired 3 presynaptic stimuli that caused 3 EPSPs (10 ms ahead) with 3 postsynaptic APs at 30 Hz, paired 15 times every 5 s (named as EPSPs-APs protocol). The third involved theta-burst stimulation (5 trains of burst with 4 pulses at 100 Hz, at 200 ms interval; repeated 4 times at intervals of 10 s; named as TBS). The NMDAR-mediated component of EPSCs was pharmacologically isolated in Mg²⁺-free ACSF containing: CNQX (20 μM), glycine (1 μM) and picrotoxin (100 μM). The patch electrodes contained (in mM) 102 cesium gluconate, 5 TEA-chloride, 3.7 NaCl, 11 BAPTA, 0.2 EGTA, 20 HEPES, 2 MgATP, 0.3 NaGTP, and 5 QX-314 chloride (adjusted to pH 7.2 with CsOH). Neurons were voltage clamped at −60 mV and NMDARs-mediated EPSCs were evoked at 0.05 Hz. Picrotoxin (100 μM) was always present to block GABA_{A} receptor-mediated inhibitory synaptic currents. The access resistance was 15-30 MΩ and was monitored throughout the experiment. Data were discarded if access resistance changed by more than 15% during an experiment. Results are expressed as means±SEM. Statistical comparisons were performed using the Student t-test.

Western Blot and Immunoprecipitation

Equal amounts of protein from the ACC and hippocampus were separated and electrotransferred onto PDVF membranes (Invitrogen), which were probed with anti-NR2A, anti-NR2B (Chemicon), and anti-PSD-95 (ABR) and with β-actin (Sigma) as a loading control. The membranes were incubated with horseradish peroxidase-conjugated secondary antibodies (anti-mouse IgG for PSD-95 and anti-rabbit IgG for the other primary antibodies), and bands were visualized using an ECL system (Perkin Elmer). Synaptosomal membrane fractions (LP1) were prepared as previously described (Dunah and Standaert, 2001) and solubilized using 1% SDS in TEVP buffer: 10 mM Tris-HCl, pH 7.4, 1 mM EDTA, 1 mM EGTA, 1× protease inhibitor cocktail (Sigma), and 1× phosphatase inhibitor cocktail 1 and 2 (Sigma). The solubilized proteins were diluted 20 fold with modified RIPA buffer (50 mM Tris-HCl, pH 7.4, 1% NP-40, 0.25% Na-deoxycholate, 150 mM NaCl, 1 mM EDTA, 1 mM PMSF), and incubated with 50 μl of protein G-agarose precoupled with anti-phosphotyrosine antibody (PY20, BD Biosciences) for 3 h at 4° C. The reaction mixtures were then washed three times and eluted by boiling in sample loading buffer and subjected to western blot as described above. Equal amounts of synaptosomal membrane fraction from the ACC and hippocampus were used for the western blotting of synaptosomal NMDA receptors. Results are expressed as means±SEM. Statistical comparisons were performed using the t-test or the paired t-test.

Microelectroporation of siRNA

siRNA corresponding to NR2B (sense 5′-GGAUGAGUCCUCCAUGUUCtt-3′ and antisense 5′-GAACAUGGAGGACUCAUCCtt-3′, Silencer Pre-designed siRNA, ID #61879, Ambion, Tex.) was delivered into the ACC by microelectroporation, which was performed as described previously (Wei et al., 2003). Briefly, 0.5 μl of 1:4 mixture of siRNA (2.5 μg/μl) and pEGFP-N1 (1 μg/μl) was injected into the ACC (0.7 mm anterior to Bregma, ±0.4 mm lateral from the midline, 1.8 mm beneath the surface of the skull) (Franklin and Paxinos, 1997) through a 30-gauge injection cannula. Then, square-wave electric pulses (five pulses at 1 Hz, 50 ms duration at 40 V) were delivered through a pair of silver electrodes placed 2 mm anterior and 1 mm posterior to the injection site using a S48 Stimulator (Grass Instruments). Control groups consisted of a group receiving only the electroporation shock, pEGFP-N1, or Silencer Negative control siRNA (#4611, Ambion) and pEGFP-N1. Western blot analysis was performed 4 days after electroporation and electrophysiological recordings were taken 2-4 days after.

Mouse Surgery and Fear Conditioning

Under ketamine and xylazine anesthesia, 24-guage guide cannulas were implanted bilaterally into the ACC (0.7 mm anterior to Bregma, ±0.4 mm lateral from the midline, 1.7 mm beneath the surface of the skull) or dorsal hippocampus (2.0 mm posterior to Bregma, ±1.5 mm lateral from the midline, 1.9 mm beneath the surface of the skull). Mice were given at least 2 weeks to recover after cannula implantation. All procedures were performed in accordance with the requirements of the Animal Studies Committee at the University of Toronto. The 30-gauge injection cannula was 0.1 mm lower than the guide. For intra-ACC infusion, 0.5 μl Ro25-6981 (4 μg/μl) or saline was delivered bilaterally within 90 s using a pump. 15 min later mice were conditioned by one pairing of a tone (2.8 kHz, 85 dB, 30 s) and a foot shock (0.75 mA, 2 s) that terminated at the same time as the tone. For intra-hippocampal infusion, 0.5 μl Ro25-6981 (10 μg/μl) or saline was delivered bilaterally within 90 s. 15 min later mice received one pairing of a tone and a foot shock, as above. One day later, animals were exposed to the conditioning context without a tone for 3 min and freezing responses were scored automatically (Freeze view software, Actrimetrics, Wilmette, Ill.).

Rat Surgery and Fear Conditioning

For one site drug infusion, 23-guage guide cannulas were implanted bilaterally into the dorsal hippocampus (CA1 region: 3.2 mm posterior to Bregma, ±1.5˜1.7 mm lateral from the midline, 1.7 mm beneath the surface of the skull). For two sites drug infusion, guide cannulas were implanted bilaterally into the dorsal hippocampus (first injection site: 2.8 mm posterior to Bregma, ±1.5 mm lateral from the midline, 1.7 mm beneath the surface of the skull; second injection site: 3.8 mm posterior to Bregma, ±1.8˜2.2 mm lateral from the midline, 1.3 mm beneath the surface of the skull). The dummy cannulas, cut 0.5 mm longer than guide cannulas, were inserted into the guide cannulas to prevent clogging and reduce the risk of infection. Rats were given at least 5 days to recover before experimentation. A 30-gauge injection cannula that was 1.5 mm lower than the guide was used for intra-hippocampal infusion. 1.0 μl Ro25-6981 (0.1 μg /μl) or saline was delivered bilaterally at the rate of 0.5 μl/min using a pump. After infusion, the cannulas were left in place for an additional 2 min to allow the solution to diffuse away from the cannula tip. Fifteen minutes later, rats were conditioned by one pairing of a tone (2.2 kHz, 96 dB, 30 s) and a foot shock (2.0 mA, 2 s) that terminated at the same time as the tone. Approximately 2 days after conditioning, contextual and auditory memory tests were conducted. Rats were returned to the conditioning chamber and allowed to stay in the chamber for 3-min without footshock.

Histological Identification

To confirm the locations of the intra-ACC and hippocampal injection sites, brains were fixed with 4% paraformaldehyde and dehydrated through an ascending alcohol series. The mice coronal sections (30 μm) were mounted on glass slides and stained with hematoxylin and eosin. The rat brains were cut into 40˜50 μm coronal sections and stained with neural red. Images were taken using an Olympus light microscope equipped with a CCD camera.

Results

Cingulate LTP in Adult Mice Requires NMDA Receptor Activation

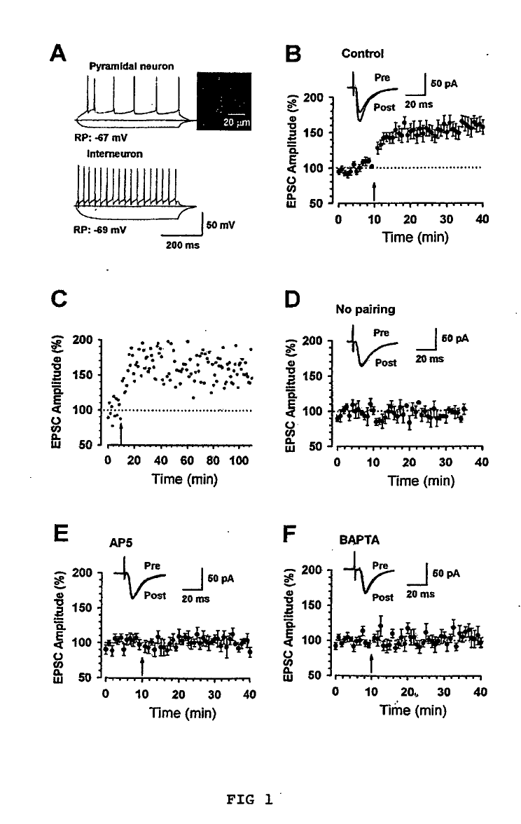

Whole-cell patch-clamp recordings were conducted in visually identified pyramidal neurons in layer II/III of ACC slices. Pyramidal neurons were identified by injecting depolarized currents into neurons to induce action potentials. The typical firing pattern of pyramidal neurons showed significant firing frequency adaptation (Tsvetkov et al., 2002), whereas interneurons showed fast-spiking action potentials followed by pronounced hyperpolarization. Pyramidal neurons were also identified based on the pyramid shape of their somata by putting Lucifer yellow into the intracellular solution (FIG. 1A). To determine if synaptic transmission undergoes LTP, synaptic stimulation was paired with postsynaptic depolarization (also referred to as ‘pairing training’) (80 pulses of presynaptic stimulation at 2 Hz in layer V with postsynaptic depolarization at +30 mV) (Artola et al., 1990; Tsvetkov et al., 2004). The pairing training produced a significant, long-lasting potentiation of synaptic responses (mean 160.0±10.8% of baseline, n=15; t-test, p<0.001 vs. baseline responses before the pairing training; FIG. 1B). In some neurons (n=5), which were recorded over a long period, LTP persisted for at least 90 minutes (FIG. 1C). In the control group, neurons were not subjected to pairing training, and synaptic responses were not significantly altered over the entire recording period (last 5 min mean 98.1±5.8% of first 5 min baseline response, n=5; t-test, p=0.66; FIG. 1D).

To determine if NMDAR activation is required for cingulate LTP induction, a selective NMDAR antagonist, AP-5 (50 μM) was applied. LTP was completely blocked (97.8±5.7%, n=7; FIG. 1E). Furthermore, LTP induction was completely abolished by 11 mM BAPTA in the pipette solution (106.1±10.3%, n=7; FIG. 1F), indicating that cingulate LTP is dependent on the activation of NMDARs and elevated postsynaptic Ca²⁺ concentrations.

Cingulate LTP Expression Depends on a Postsynaptic Mechanism

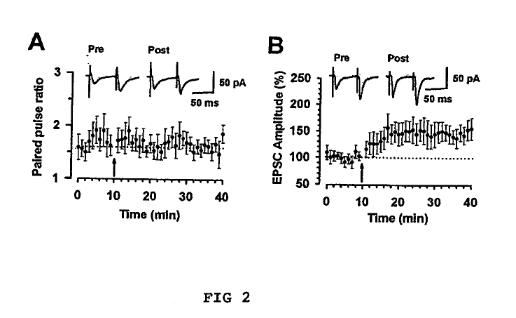

Both presynaptic and postsynaptic mechanisms have been proposed to contribute to LTP expression (Nicoll and Malenka, 1995). For example, in the CA3 region the presynaptic expression of LTP is accompanied by altered paired-pulse facilitation (PPF) (Zalutsky and Nicoll, 1990). To determine whether presynaptic mechanisms are involved in expression of LTP in the ACC, PPF was measured before and after LTP induction. As shown in FIG. 2A, PPF was not altered after the LTP-induction protocol (ratios of EPSC2/EPSC1 1.8±0.2 and 1.6±0.2, respectively, before and 25 min after LTP induction, n=5; paired t-test, p=0.39), whereas synaptic responses were significantly enhanced in the same slices (EPSC1 149.0±17.0% of baseline, n=5; t-test, p<0.05 vs. baseline; FIG. 2B). To determine the possible role of presynaptic mechanisms in ACC LTP expression, NMDAR-mediated currents were tested to determine whether they were altered after LTP induction. Although NMDAR-mediated currents were expected to be enhanced if presynaptic mechanisms were involved, LTP induced NMDAR-mediated currents were decreased to 76.8±5.0% of baseline (n=7; t-test, p<0.01; FIG. S1). This result suggests that the expression of LTP in the ACC depends on a postsynaptic mechanism.

Requirement of NR2A and NR2B Subunits

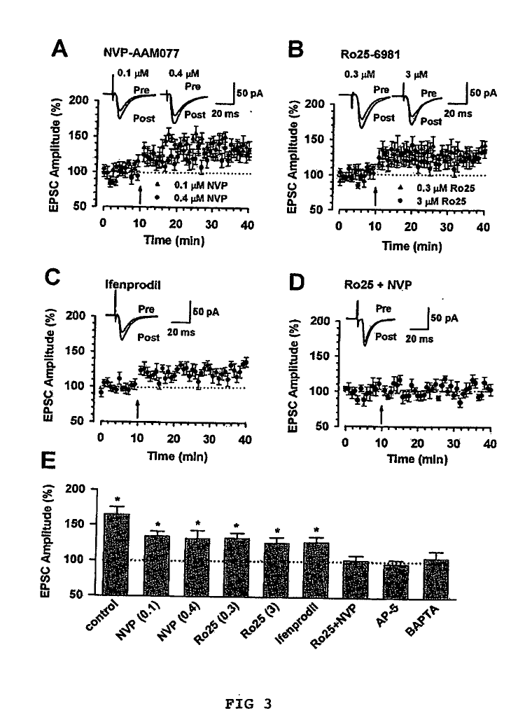

The contribution of both NR2A and NR2B to the induction of cingulate LTP was examined by application of the specific NR2A subunit antagonist, NVP-AAM077 (IC50 of 14 nM and 1.8 μM for NR1/NR2A and NR1/NR2B, respectively) (Auberson et al., 2002), and the NR2B subunit antagonist, Ro25-6981 (IC50 of 9 nM and 52 μM for NR1/NR2B and NR1/NR2A respectively) (Fischer et al., 1997) or ifenprodil (IC50 of 0.34 μM and 146 μM for NR1/NR2B and NR1/NR2A respectively) (Williams, 1993). As shown in FIG. 3A, cingulate LTP was significantly reduced but not completely blocked by two different doses of NVP-AAM077 (0.1 μM: 134.9±6.9%, n=7; t-test, p<0.01; 0.4 μM: 131.6±11.9%, n=9; t-test, p<0.01). This partial blockade of LTP raises the possibility that the NR2B subunit contributes to the induction of cingulate LTP. The effects of NR2B subunit selective antagonists on the induction of LTP was then examined. LTP was partially reduced by two doses of Ro25-6981 (0.3 μM: 132.8±7.4%, n=9; p<0.01; 3 μM: 126.8±7.5%, n=5; p<0.05; FIG. 3B) or 3 μM ifenprodil (128.1±7.4%, n=7; p<0.05; FIG. 3C). Because NR2A or NR2B antagonists only partially blocked LTP, the effects of combinations of the two antagonists was tested. LTP was completely blocked by the combination of 0.4 μM NVP-AAM077 and 0.3 μM Ro 25-6981 (102.7±7.1%, n=6; FIG. 3D). These results provide evidence that both NR2B and NR2A NMDAR subunits contribute to the formation of cingulate LTP.

Involvement of the NR2B Subunit is Not Dependent on LTP Induction Protocols

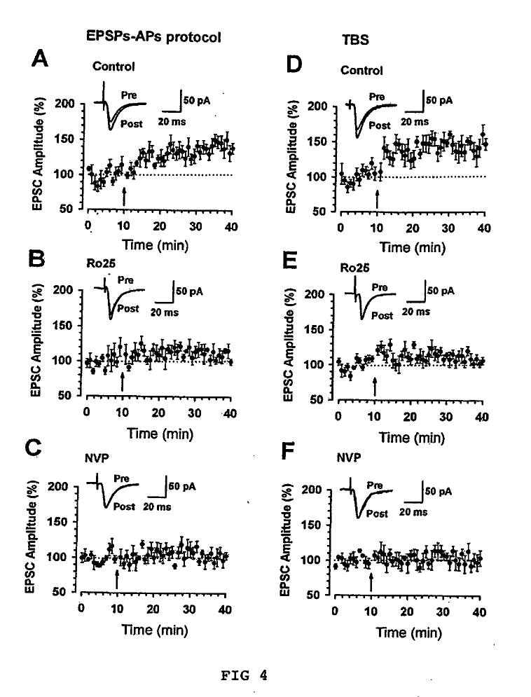

To test whether the involvement of NR2A and NR2B subunits is dependent on the specific LTP induction paradigm used, the role of NR2A and NR2B using two different induction protocols was examined. First, a protocol (EPSPs-APs protocol, see methods) based on the coincidence of postsynaptic action potentials (APs) and unitary excitatory postsynaptic potentials (EPSPs, 10 ms ahead) to induce LTP (Bi and Poo, 1998; Markram et al., 1997) was used. It was found that this protocol produced a significant, long-lasting potentiation (141.9±11.9%, n=5; t-test, p<0.05 vs. baseline; FIG. 4A). Moreover, this potentiation was completely blocked by either 0.3 μM Ro25-6981 (114.4±11.5%, n=5) or 0.4 μM NVP-AAM077 (102.0±11.5%, n=5; FIGS. 4B-C). Second, LTP was induced using theta-burst stimulation (TBS). It was found that TBS induced a significant LTP in the ACC (144.1±11.8%; n=5; t-test, p<0.05; FIG. 4D). Similarly, LTP was blocked by 0.3 μM Ro25-6981 (105.5±8.3%, n=7) or 0.4 μM NVP-AAM077 (97.0±13.4%, n=5; FIGS. 4E-F). Taken together, these results indicate that the role of NR2B in the induction of LTP does not depend on the induction paradigm.

NR2A and NR2B Mediated EPSCs in the ACC and Hippocampus

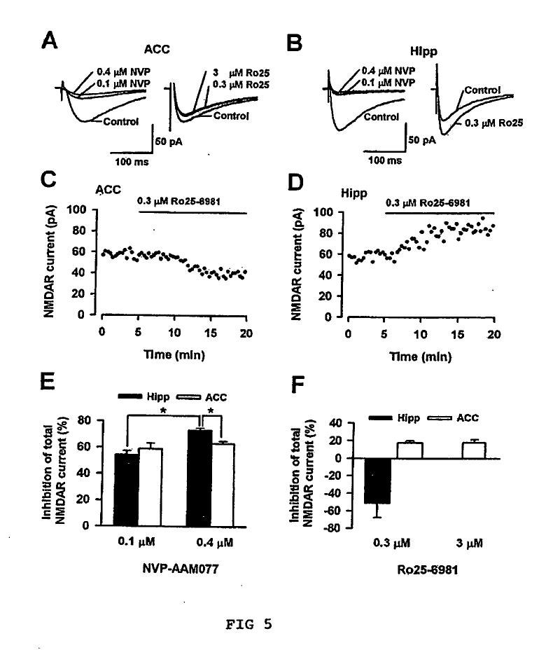

It has been shown that the NMDA NR2B receptors are not responsible for LTP induction in the hippocampus (Liu et al., 2004) indicating that different mechanisms underly the different roles of NR2B in cingulate and hippocampal LTP. One possible mechanism is that NR2B-containing receptors may contribute more to total NMDA currents in ACC synapses than in hippocampal synapses. To test this, pharmacological antagonists for NR2A or NR2B were used to examine synaptically induced NMDAR-mediated EPSCs. Application of 0.1 μM or 0.4 μM NVP-AAM077 depressed total NMDAR-mediated currents by 59.0±4.3% (n=5) and 63.2±1.7% (n=6) in the ACC respectively (FIG. 5). On the other hand, application of 0.3 μM or 3 μM Ro25-6981 attenuated total NMDAR-mediated currents by 18.0±1.9% (n=5) and 18.1±3.4% (n=7) in the ACC respectively, showing that 0.3 μM Ro25-6981 was sufficient to block the NR2B-mediated currents (FIG. 5). This experiment was repeated in hippocampal CA1 neurons. In this case, it was found that 0.1 μM or 0.4 μM NVP-AAM077 depressed total NMDAR-mediated currents by 54.4±3.2% (n=5) and 73.1±1.7% (n=6) respectively (p<0.001, FIGS. 5B-E). However, unlike in the ACC, application of 0.3 μM Ro25-6981 alone potentiated, rather than blocked, total NMDAR-mediated currents by 51.3±16.4% in the hippocampus (FIG. 5). This result is consistent with a recent report in the adult rat hippocampus suggesting an inhibitory relationship between NR2B and NR2A-subunit containing NMDARs (Mallon et al., 2005).

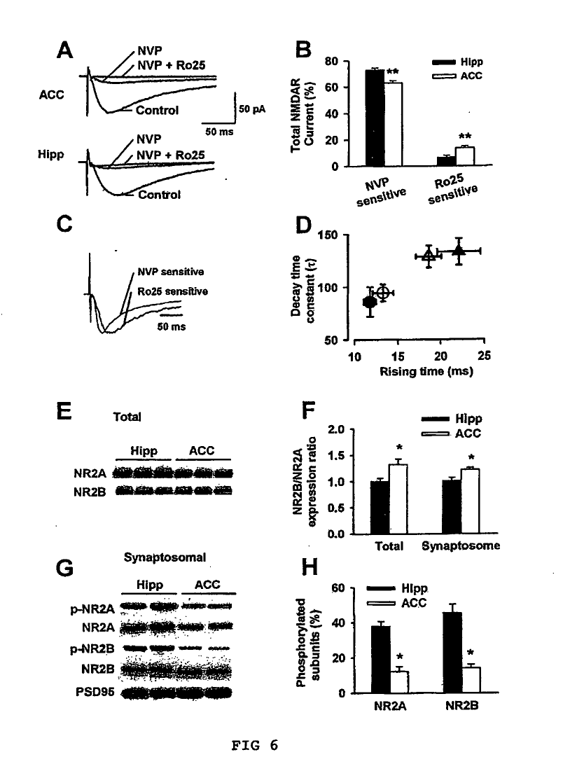

The inconsistent effect of Ro25-6981 on NMDAR-mediated currents between the ACC and hippocampus indicates that distinctive NMDAR properties or differential expression of NR2B and NR2A proteins may exist or occur in these two regions. The relative percentages of NR2A- and NR2B-mediated currents were determined by applying specific antagonists, NVP-AAM077 (0.4 μM) and Ro25-6981 (0.3 μM). The concentrations used here were based on the results from FIG. 5 and a recent study performed in the hippocampus (Liu et al., 2004). Application of NVP-AAMO77 depressed total NMDAR-mediated currents by 63.2±1.7% and the addition of Ro25-6981 to the same neuron further reduced currents by 13.7±1.4% (n=6) in the ACC (FIG. 6). Reversing this order, by first applying Ro25-6981 and then adding NVP-AAM077, resulted in total NMDAR-mediated currents being depressed by 18.0±1.9% and 64.2±3.0% (n=6), respectively. Since Ro25-6981 and NVP-AAM077 show a similar effect on NMDAR-mediated currents regardless of the order of application, it is appropriate to use these antagonists to study NMDAR-mediated currents within the ACC, however, the possibility that these antagonists also block triheteromers of NMDARs (NR1/2A/2B) cannot be excluded (Hatton and Paoletti, 2005).

The same experiment was repeated in the hippocampus. By first applying NVP-AAM077 (0.4 μM) and then adding Ro25-6981 (0.3 μM) to the same neuron in a hippocampal sample, the inhibitory effect of 0.4 μM NVP-AAM077 in the hippocampus (73.1±1.7%, n=6) was found to be significantly greater than that in the ACC neurons (63.2±1.7%, n=6; t-test, p<0.01; FIG. 6B). The application of 0.3 μM Ro25-6981 produced a further reduction in the hippocampus that was significantly smaller when compared to the inhibitory effect obtained in the ACC (ACC: 13.7±1.4%, n=6; vs. hippocampus: 6.6±1.4%, n=6, t-test, p<0.01; FIG. 6B). Kinetic analysis showed that the mean rise time and decay constants (τ) of NR2B-mediated NMDAR currents were greater than those of NR2A-mediated NMDAR currents, but these characteristics were similar in the ACC and hippocampus (FIGS. 6C-D).

Expression and Phosphorylation of the NR2B and NR2A Receptors

The different contributions made by NR2B and NR2A were then examined to determine if the differences were due to differential expression of NR2B and NR2A proteins in the ACC and hippocampus. Relative subunit expression ratios of NR2B to NR2A in the ACC and hippocampus were examined by western blot analysis on total homogenates or synaptosomal membrane fractions. Western blot results showed that the basal expression level of NR2A was consistently higher in the hippocampus compared to the ACC. In contrast, NR2B subunit expression was similar in both tissues (FIG. 6E). In addition, the expression of PSD-95, a postsynaptic marker protein, was similar in the two tissues (FIG. 6G). Thus the NR2B/NR2A ratio in total homogenates was higher in the ACC than in the hippocampus (n=7; p<0.05; FIG. 6F). Similarly, in synaptosomal membranes, the NR2B/NR2A ratio was also found to be higher in the ACC than in the hippocampus (n=4; p<0.05; FIG. 6F). Thus, the higher contribution of NR2B in ACC synapses could explain the role of NR2B in LTP induction.

The function of NMDARs is regulated by its phosphorylation. Since tyrosine phosphorylation of NMDARs is thought to contribute to LTP (Collingridge and Singer, 1990; Lu et al., 1998), tyrosine phosphorylation levels of NR2A and NR2B subunits with anti-phosphotyrosine antibody was examined to determine whether NMDARs are phosphorylated to different extents in the ACC and hippocampus (FIG. 6G). NR2B subunits are found both intra-and extra-synaptically where they are involved in induction of LTD (Massey et al., 2004); however, since the current study focuses on the role of NR2A and NR2B subunits in LTP phosphorylation levels in synaptosomal membrane fractions were examined. Tyrosine phosphorylation levels of both NR2A and NR2B in the ACC (n=4) were found to be significantly lower than in the hippocampus (n=4; p<0.01; FIG. 6H).

Reduction of NR2B Expression by siRNA Electroporation

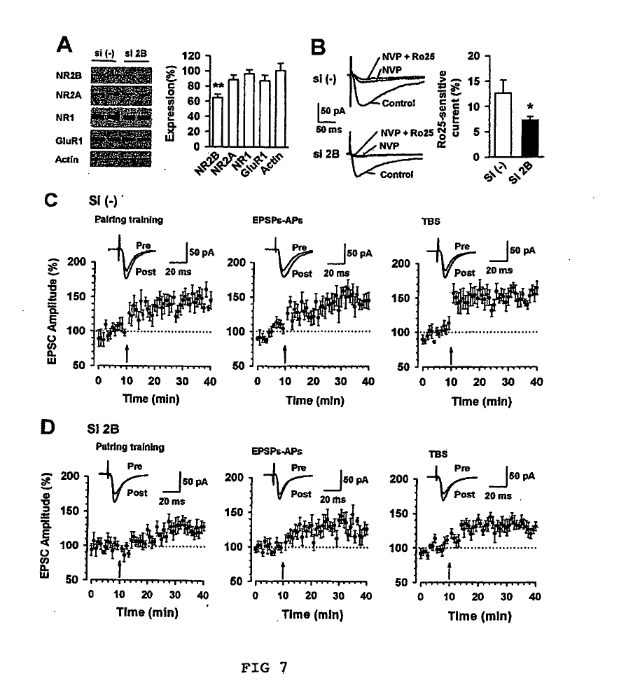

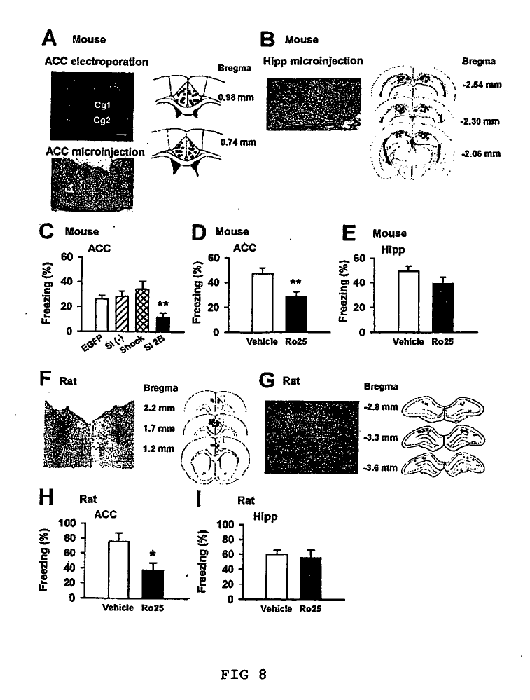

Since LTP is believed to be an underlying mechanism in many forms of synaptic plasticity and memory, including fear conditioning mediated by the amygdala (Rodrigues et al., 2004; Rogan et al., 1997) and because the present results demonstrate that the NR2B subunit of NMDA receptors is required for ACC LTP induction, the role of cortical NR2B in contextual fear conditioning (Rodrigues et al., 2004) was examined. To inhibit NR2B expression specifically in the prefrontal cortex, including the ACC, siRNA was bilaterally delivered against NR2B with a reporter plasmid encoding EGFP by microelectroporation (Wei et al., 2003) (FIG. 8A). Two different experimental approaches were used to confirm the effectiveness and specificity of the NR2B siRNA. First, we performed western blot analysis for NR2B and other membrane proteins. The expression levels of other NMDAR subunits (NR2A and NR1) and AMPA receptor subunit GluR1 were measured, and actin was used as a control in the ACCs of NR2B siRNA and control siRNA electroporated mice. We found that only NR2B protein was significantly reduced four days after electroporation (64.8±4.6% of NR2B expression compared to control siRNA electroporated control; n=5 mice, p<0.01; FIG. 7A). The expression levels of NR2A, NR1, GluR1, and actin were not significantly changed.

NMDAR-mediated EPSCs were also recorded in neurons from electroporated ACC slices. Consistently, NR2B-mediated NMDA EPSCs were significantly reduced in NR2B siRNA-treated neurons (7.3±0.7%, n=11 neurons from 5 mice vs. control slices, 12.6±2.6% of total NMDAR-mediated current, n=5 neurons from 2 mice; p<0.05; FIG. 7B). Taken together, the data suggests that the effect of NR2B siRNA electroporation is target-specific in terms of both protein expression and receptor function.

Reduced LTP by NR2B siRNA Electroporation

Having confirmed that NR2B expression was reduced in NR2B siRNA-treated neurons, whether NR2B siRNA impairs the induction of LTP in ACC slices was examined using different induction protocols. A pairing training protocol was used to induce LTP and found that potentiation was significantly reduced in NR2B siRNA-treated neurons as compared to neurons in control siRNA electroporated mice (n=6 for each group; FIGS. 7C-D). Second, TBS-induced potentiation was also significantly reduced (n=6, p<0.05; FIG. 7). A similar reduction was found in LTP induced by EPSPs-APs protocol (n=6, p<0.05; FIG. 7). LTP, induced by three different protocols, did not differ in mice injected with control siRNA compared to mice that did not receive an injection (p>0.05).

Genetic and Pharmacological Inhibition of Cortical NR2B Impairs Contextual Fear Memory Formation

NR2B subunit inhibition in adult mice was examined to determine whether this impairs the formation of contextual fear memory. Three days after bilateral siRNA electroporation, mice were trained (or conditioned) and then tested for contextual fear memory by assessing freezing behavior in the same environmental context after 24 hours. There was no difference in baseline freezing or in freezing immediately following the shock/tone pairing between groups; however, there was a significant difference between groups when tested in the contextual environment one day later (n=8 for EGFP only; n=10 for control siRNA; n=8 for shock only; n=11 for NR2B siRNA; one-way ANOVA, p=0.005; FIG. 8C). NR2B siRNA injected mice displayed significantly less freezing compared to GFP (p<0.05), shock only (p<0.01) and control siRNA group (p<0.05), which suggests that NR2B in the cortex is involved in contextual fear memory processing. However, auditory fear memory was similar between all treatment groups (one-way ANOVA, p=0.43). Since the activation of NR2B was suppressed throughout the training and memory test, the roles of NR2B during the acquisition and retrieval of fear memory cannot be distinguished. Recently, electric stimulation of the ACC was reported to induce fear memory one and three days after stimulation (Tang et al., 2005), which implies that the ACC may be involved in the acquisition of fear memory. To directly address this issue, the effect of NR2B blockade using Ro25-6981 was examined. Microinjection of Ro25-6981 (2 μg in 0.5 μl per side) into the bilateral ACC before conditioning produced a significant reduction in freezing in the contextual environment (Ro25-6981, n=11; vehicle, n=10, p<0.01; FIG. 8D). There was no difference in auditory fear memory between Ro25-6981 and vehicle injected mice (p=0.65). In order to rule out any locomotor side-effects, locomotor activity in the open field was tested after bilateral ACC microinjection of Ro25-6981 (n=4) or vehicle (n=4). There was no difference in locomotor activity between groups when recorded 15 minutes or one day after injection. Taken together, this suggests that the activation of NR2B in the ACC is important for the acquisition of contextual fear memory.

The hippocampus plays a role in contextual memory and intra-hippocampal injections of the NMDAR antagonist MK-801, which blocks NMDAR-mediated signaling, resulted in reduced contextual freezing (Bast et al., 2003; Fanselow, 2000; Fanselow et al., 1994). However, the effects of local hippocampal injections of NR2B antagonists on fear conditioning have not been examined. Ro25-6981 (5 μg in 0.5 μl) was bilaterally injected into dorsal hippocampi (FIG. 8B) before conditional training. As in the ACC, hippocampal microinjection did not significantly affect baseline or immediate freezing behavior. Selective blockade of the NR2B subunit did not affect contextual or auditory fear memory when tested one day after training (contextual: Ro25-6981, n=12; vehicle, n=9; FIG. 8E; auditory: p=0.08). The NR2B specific antagonist was also injected into the ACC and hippocampus of rats and contextual and auditory fear memory was then measured. Similar to the results from mice, microinjection of Ro25-6981 (2 g in 1 μl per side) bilaterally into the ACC (FIG. 8F) before conditioning, produced a significant reduction in contextual fear memory (n=6 for each group, p<0.05; FIG. 8H) when tested one day after training. However, intra-hippocampal infusion of Ro25-6981 (0.1 μg in 1 μl) bilaterally into dorsal hippocampus (FIG. 8G) before fear conditioning did not produce a significant impairment in the expression of contextual memory (Ro25-6981, n=6; vehicle, n=7; p=0.67; FIG. 8I). Consistent with results in mice, Ro25-6981 did not affect auditory fear memory when injected in either the ACC or hippocampus (ACC: Ro25-6981, n=6; vehicle, n=6; p=0.65; Hippocampus: Ro25-6981, n=6; vehicle, n=7; p=0.78; data not shown). Hippocampal injections of a higher dose of Ro25-6981 (5 μg in 1 μl per side) bilaterally or multiple injections of the same dose (2 per side) were then tested to determine their affect on either contextual or auditory fear memory. Neither single nor multiple injections of Ro25-6981 produced a significant impairment in the expression of contextual or auditory fear memory (FIG. S2).

Discussion

The present data indicate that both NMDAR NR2A and NR2B subunits are involved in the formation of LTP in the ACC, a central region of the brain that is important for cognitive function. Furthermore, it was determined, based on results obtained by electrophysiological, pharmacological, behavioural and genetic approaches, that NR2B in the ACC is specifically involved in the formation of contextual fear memory, and that inhibition of NR2B prevents or at least reduces the occurrence of contextual fear memory, providing a novel method for the treatment of fear-related conditions.

The foregoing examples exemplify only certain embodiments of the invention. As one of skill in the art will appreciate, other embodiments of the invention are possible that fall within the scope of the appended claims. All references made herein are incorporated by reference.

### REFERENCES

Arrigoni, E., and Greene, R. W. (2004). Schaffer collateral and perforant path inputs activate different subtypes of NMDA receptors on the same CA1 pyramidal cell. Br J Pharmacol 142, 317-322.
Artola, A., Brocher, S., and Singer, W. (1990). Different voltage-dependent thresholds for inducing long-term depression and long-term potentiation in slices of rat visual cortex. Nature 347, 69-72.
Auberson, Y. P., Allgeier, H., Bischoff, S., Lingenhoehl, K., Moretti, R., and Schmutz, M. (2002). 5-Phosphonomethylquinoxalinediones as competitive NMDA receptor antagonists with a preference for the human 1A/2A, rather than 1A/2B receptor composition. Bioorg Med Chem Lett 12, 1099-1102.
Bast, T., Zhang, W. N., and Feldon, J. (2003). Dorsal hippocampus and classical fear conditioning to tone and context in rats: effects of local NMDA-receptor blockade and stimulation. Hippocampus 13, 657-675.
Bear, M. F., and Kirkwood, A. (1993). Neocortical long-term potentiation. Curr Opin Neurobiol 3, 197-202.
Bi, G. Q., and Poo, M. M. (1998). Synaptic modifications in cultured hippocampal neurons: dependence on spike timing, synaptic strength, and postsynaptic cell type. J Neurosci 18, 10464-10472.
Bliss, T. V., and Collingridge, G. L. (1993). A synaptic model of memory: long-term potentiation in the hippocampus. Nature 361, 31-39.
Cassell, M. D., and Wright, D. J. (1986). Topography of projections from the medial prefrontal cortex to the amygdala in the rat. Brain Res Bull 17, 321-333.
Collingridge, G. L., and Singer, W. (1990). Excitatory amino acid receptors and synaptic plasticity. Trends Pharmacol Sci 11, 290-296.
Cui, Z., Wang, H., Tan, Y., Zaia, K. A., Zhang, S., and Tsien, J. Z. (2004). Inducible and reversible NR1 knockout reveals crucial role of the NMDA receptor in preserving remote memories in the brain. Neuron 41, 781-793.
Devinsky, O., Morrell, M. J., and Vogt, B. A. (1995). Contributions of anterior cingulate cortex to behaviour. Brain 118 (Pt 1), 279-306.
Dunah, A. W., and Standaert, D. G. (2001). Dopamine D1 receptor-dependent trafficking of striatal NMDA glutamate receptors to the postsynaptic membrane. J Neurosci 21, 5546-5558.
Fanselow, M. S. (2000). Contextual fear, gestalt memories, and the hippocampus. Behav Brain Res 110, 73-81.
Fanselow, M. S., Kim, J. J., Yipp, J., and De Oca, B. (1994). Differential effects of the N-methyl-D-aspartate antagonist DL-2-amino-5-phosphonovalerate on acquisition of fear of auditory and contextual cues. Behav Neurosci 108, 235-240.
Fischer, G., Mutel, V., Trube, G., Malherbe, P., Kew, J. N., Mohacsi, E., Heitz, M. P., and Kemp, J. A. (1997). Ro 25-6981, a highly potent and selective blocker of N-methyl-D-aspartate receptors containing the NR2B subunit. Characterization in vitro. J Pharmacol Exp Ther 283, 1285-1292.
Frankland, P. W., Bontempi, B., Talton, L. E., Kaczmarek, L., and Silva, A. J. (2004). The involvement of the anterior cingulate cortex in remote contextual fear memory. Science 304, 881-883.
Frankland, P. W., O'Brien, C., Ohno, M., Kirkwood, A., and Silva, A. J. (2001). Alpha-CaMKII-dependent plasticity in the cortex is required for permanent memory. Nature 411, 309-313.
Franklin, K. B. J., and Paxinos, G. (1997). The Mouse Brain in Stereotaxic Coordinates (New York, Academic Press).
Gao, Y. J., Ren, W. H., Zhang, Y. Q., and Zhao, Z. Q. (2004). Contributions of the anterior cingulate cortex and amygdala to pain- and fear-conditioned place avoidance in rats. Pain 110, 343-353.
Gewirtz, J. C., Falls, W. A., and Davis, M. (1997). Normal conditioned inhibition and extinction of freezing and fear-potentiated startle following electrolytic lesions of medical prefrontal cortex in rats. Behav Neurosci 111, 712-726.
Han, C. J., O'Tuathaigh, C. M., van Trigt, L., Quinn, J. J., Fanselow, M. S., Mongeau, R., Koch, C., and Anderson, D. J. (2003). Trace but not delay fear conditioning requires attention and the anterior cingulate cortex. Proc Natl Acad Sci USA 100, 13087-13092.
Hatton, C. J., and Paoletti, P. (2005). Modulation of triheteromeric NMDA receptors by N-terminal domain ligands. Neuron 46, 261-274.
Hayashi, M. L., Choi, S. Y., Rao, B. S., Jung, H. Y., Lee, H. K., Zhang, D., Chattaiji, S., Kirkwood, A., and Tonegawa, S. (2004). Altered cortical synaptic morphology and impaired memory consolidation in forebrain-specific dominant-negative PAK transgenic mice. Neuron 42, 773-787.
Johansen, J. P., and Fields, H. L. (2004). Glutamatergic activation of anterior cingulate cortex produces an aversive teaching signal. Nat Neurosci 7, 398-403.
Kalia, L. V., Gingrich, J. R., and Salter, M. W. (2004). Src in synaptic transmission and plasticity. Oncogene 23, 8007-8016.
Kiyama, Y., Manabe, T., Sakimura, K., Kawakami, F., Mori, H., and Mishina, M. (1998). Increased thresholds for long-term potentiation and contextual learning in mice lacking the NMDA-type glutamate receptor epsilon1 subunit. J Neurosci 18, 6704-6712.
Knight, D. C., Cheng, D. T., Smith, C. N., Stein, E. A., and Helmstetter, F. J. (2004). Neural substrates mediating human delay and trace fear conditioning. J Neurosci 24, 218-228.
Kohr, G., Jensen, V., Koester, H. J., Mihaljevic, A. L., Utvik, J. K., Kvello, A., Ottersen, O. P., Seeburg, P. H., Sprengel, R., and Hvalby, O. (2003). Intracellular domains of NMDA receptor subtypes are determinants for long-term potentiation induction. J Neurosci 23, 10791-10799.
Lisman, J. (2003). Long-term potentiation: outstanding questions and attempted synthesis. Philos Trans R Soc Lond B Biol Sci 358, 829-842.
Liu, L., Wong, T. P., Pozza, M. F., Lingenhoehl, K., Wang, Y., Sheng, M., Auberson, Y. P., and Wang, Y. T. (2004). Role of NMDA receptor subtypes in governing the direction of hippocampal synaptic plasticity. Science 304, 1021-1024.
Loftis, J. M., and Janowsky, A. (2003). The N-methyl-D-aspartate receptor subunit NR2B: localization, functional properties, regulation, and clinical implications. Pharmacol Ther 97, 55-85.
Lu, Y. M., Roder, J. C., Davidow, J., and Salter, M. W. (1998). Src activation in the induction of long-term potentiation in CA1 hippocampal neurons. Science 279, 1363-1367.
Malenka, R. C., and Nicoll, R. A. (1993). NMDA-receptor-dependent synaptic plasticity: multiple forms and mechanisms. Trends Neurosci 16, 521-527.
Mallon, A. P., Auberson, Y. P., and Stone, T. W. (2005). Selective subunit antagonists suggest an inhibitory relationship between NR2B and NR2A-subunit containing N-methyl-D:-aspartate receptors in hippocampal slices. Exp Brain Res 162, 374-383.
Markram, H., Lubke, J., Frotscher, M., and Sakmann, B. (1997). Regulation of synaptic efficacy by coincidence of postsynaptic APs and EPSPs. Science 275, 213-215.
Massey, P. V., Johnson, B. E., Moult, P. R., Auberson, Y. P., Brown, M. W., Molnar, E., Collingridge, G. L., and Bashir, Z. I. (2004). Differential roles of NR2A and NR2B-containing NMDA receptors in cortical long-term potentiation and long-term depression. J Neurosci 24, 7821-7828.
Maviel, T., Durkin, T. P., Menzaghi, F., and Bontempi, B. (2004). Sites of neocortical reorganization critical for remote spatial memory. Science 305, 96-99.
McHugh, T. J., Blum, K. I., Tsien, J. Z., Tonegawa, S., and Wilson, M. A. (1996). Impaired hippocampal representation of space in CA1-specific NMDAR1 knockout mice. Cell 87, 1339-1349.
Milad, M. R., and Quirk, G. J. (2002). Neurons in medial prefrontal cortex signal memory for fear extinction. Nature 420, 70-74.
Monyer, H., Burnashev, N., Laurie, D. J., Sakmann, B., and Seeburg, P. H. (1994). Developmental and regional expression in the rat brain and functional properties of four NMDA receptors. Neuron 12, 529-540.
Morgan, M. A., and LeDoux, J. E. (1999). Contribution of ventrolateral prefrontal cortex to the acquisition and extinction of conditioned fear in rats. Neurobiol Learn Mem 72, 244-251.
Morrow, B. A., Elsworth, J. D., Inglis, F. M., and Roth, R. H. (1999). An antisense oligonucleotide reverses the footshock-induced expression of fos in the rat medial prefrontal cortex and the subsequent expression of conditioned fear-induced immobility. J Neurosci 19, 5666-5673.
Munoz, A., Woods, T. M., and Jones, E. G. (1999). Laminar and cellular distribution of AMPA, kainate, and NMDA receptor subunits in monkey sensory-motor cortex. J Comp Neurol 407, 472-490.
Nicoll, R. A., and Malenka, R. C. (1995). Contrasting properties of two forms of long-term potentiation in the hippocampus. Nature 377, 115-118.
Ritter, L. M., Vazquez, D. M., and Meador-Woodruff, J. H. (2002). Ontogeny of ionotropic glutamate receptor subunit expression in the rat hippocampus. Brain Res Dev Brain Res 139, 227-236.
Rodrigues, S. M., Schafe, G. E., and LeDoux, J. E. (2001). Intra-amygdala blockade of the NR2B subunit of the NMDA receptor disrupts the acquisition but not the expression of fear conditioning. J Neurosci 21, 6889-6896.
Rodrigues, S. M., Schafe, G. E., and LeDoux, J. E. (2004). Molecular mechanisms underlying emotional learning and memory in the lateral amygdala Neuron 44, 75-91.
Rogan, M. T., Staubli, U. V., and LeDoux, J. E. (1997). Fear conditioning induces associative long-term potentiation in the amygdala. Nature 390, 604-607.
Sah, P., and Nicoll, R. A. (1991). Mechanisms underlying potentiation of synaptic transmission in rat anterior cingulate cortex in vitro. J Physiol 433, 615-630.
Santini, E., Ge, H., Ren, K., Pena de Ortiz, S., and Quirk, G. J. (2004). Consolidation of fear extinction requires protein synthesis in the medial prefrontal cortex. J Neurosci 24, 5704-5710.
Shallice, T., Fletcher, P., Frith, C. D., Grasby, P., Frackowiak, R. S., and Dolan, R. J. (1994). Brain regions associated with acquisition and retrieval of verbal episodic memory. Nature 368, 633-635.
Sheng, M., Cummings, J., Roldan, L. A., Jan, Y. N., and Jan, L. Y. (1994). Changing subunit composition of heteromeric NMDA receptors during development of rat cortex. Nature 368, 144-147.
Tang, J., Ko, S., Ding, H. K, Qiu, C.-S., Calejesan, A. A., and Zhuo, M. (2005). Pavlovian fear memory induced by activation in the anterior cingulate cortex. Molecular Pain 1, 6.
Tang, Y. P., Shimizu, E., Dube, G. R., Rampon, C., Kerchner, G. A., Zhuo, M., Liu, G., and Tsien, J. Z. (1999). Genetic enhancement of learning and memory in mice. Nature 401, 63-69.
Tsvetkov, E., Carlezon, W. A., Benes, F. M., Kandel, E. R., and Bolshakov, V. Y. (2002). Fear conditioning occludes LTP-induced presynaptic enhancement of synaptic transmission in the cortical pathway to the lateral amygdala. Neuron 34, 289-300.
Tsvetkov, E., Shin, R. M., and Bolshakov, V. Y. (2004). Glutamate uptake determines pathway specificity of long-term potentiation in the neural circuitry of fear conditioning. Neuron41, 139-151.
Wang, Y. T., and Salter, M. W. (1994). Regulation of NMDA receptors by tyrosine kinases and phosphatases. Nature 369, 233-235.
Wei, F., Li, P., and Zhuo, M. (1999). Loss of synaptic depression in mammalian anterior cingulate cortex after amputation. J Neurosci 19, 9346-9354.
Wei, F., Qiu, C. S., Liauw, J., Robinson, D. A., Ho, N., Chatila, T., and Zhuo, M. (2002). Calcium calmodulin-dependent protein kinase IV is required for fear memory. Nat Neurosci 5, 573-579.
Wei, F., Wang, G. D., Kerchner, G. A., Kim, S. J., Xu, H. M., Chen, Z. F., and Zhuo, M. (2001). Genetic enhancement of inflammatory pain by forebrain NR2B overexpression. Nat Neurosci 4, 164-169.
Wei, F., Xia, X. M., Tang, J., Ao, H., Ko, S., Liauw, J., Qiu, C. S., and Zhuo, M. (2003). Calmodulin regulates synaptic plasticity in the anterior cingulate cortex and behavioral responses: a microelectroporation study in adult rodents. J Neurosci 23, 8402-8409.
Williams, IC (1993). Ifenprodil discriminates subtypes of the N-methyl-D-aspartate receptor: selectivity and mechanisms at recombinant heteromeric receptors. Mol Pharmacol 44, 851-859.
Wiltgen, B. J., Brown, R. A., Talton, L. E., and Silva, A. J. (2004). New circuits for old memories: the role of the neocortex in consolidation. Neuron 44, 101-108.
Wong, R. W., Setou, M., Teng, J., Takei, Y., and Hirokawa, N. (2002). Overexpression of motor protein KIF17 enhances spatial and working memory in transgenic mice. Proc Natl Acad Sci USA 99, 14500-14505.
Wu, L. J., Zhao, M. G., Toyoda, H., Ko, S., and Zhuo, M. (2005). Kainate receptor-mediated synaptic transmission in the adult anterior cingulate cortex. J Neurophysiol.
Zalutsky, R. A., and Nicoll, R. A. (1990). Comparison of two forms of long-term potentiation in single hippocampal neurons. Science 248, 1619-1624.
Zhuo, M. (2002). Glutamate receptors and persistent pain: targeting forebrain NR2B subunits. Drug Discov Today 7, 259-267.

We claim:

1. A method of treating in a mammal a condition associated with contextual fear memory, comprising the step of inhibiting the NR2B receptor in the ACC of the mammal.

2. A method as defined in claim 1, wherein the NR2B receptor is inhibited by administration of an NR2B antagonist.

3. A method as defined in claim 1, wherein the NR2B receptor is inhibited by administration of nucleic acid capable of binding to the NR2B gene.

4. A method as defined in claim 3, wherein the nucleic acid is siRNA.

5. A method as defined in claim 4, wherein the siRNA is derived from the central region of the NR2B gene.

6. A method as defined in claim 5, wherein the siRNA strands comprise i) 3′-UU overhangs on sense and antisense strands, ii) comprise from about 45-55% G/C content and iiii) are derived from nucleotides 50-100 from the start codon of the NR2B gene.

7. A method as defined in claim 5, wherein the siRNA strands are selected from the group consisting of: sense strand 5′-GGAUGAGUCCUCCAUGUUCtt-3′ and antisense strand 5′-GAACAUGGAGGACUCAUCCtt-3′, and functionally equivalent strands thereof; and sense strand 5′-AGCUCGUUCCCAAAGAGCUU-3′ and anti-sense strand 3′-UUUCGAGCAAGGGUUUUCUCG-5′, and functionally equivalent strands thereof.

8. An article of manufacture comprising a labeled container within which is a composition comprising an NR2B inhibitor suitable for the selective inhibition of NR2B in the ACC of a mammal, the label indicating that the composition is suitable to treat conditions associated with contextual fear memory.

9. An article of manufacture as defined in claim 8, wherein the NR2B inhibitor is siRNA.

10. An article of manufacture as defined in claim 9, wherein the siRNA is derived from the central region of the NR2B gene.

11. An article of manufacture as defined in claim 9, wherein the siRNA strands comprise i) 3′-UU overhangs on sense and antisense strands, ii) comprise from about 45-55% G/C content and iiii) are derived from nucleotides 50-100 from the start codon of the NR2B gene.

12. An article of manufacture as defined in claim 9, wherein the siRNA strands are selected from the group consisting of: sense strand 5′-GGAUGAGUCCUCCAUGUUCtt-3′ and antisense strand 5′-GAACAUGGAGGACUCAUCCtt-3′, and functionally equivalent strands thereof; and sense strand 5′-AGCUCGUUCCCAAAGAGCUU-3′ and anti-sense strand 3′-UUUCGAGCAAGGGUUUUCUCG-5′, and functionally equivalent strands thereof.
